(19) United States
(12) Patent Application Publication
(10) Pub. No.: US 2012/0053402 A1
(43) Pub. Date: Mar. 1, 2012

(54) MINIMALLY INVASIVE SURGERY

(76) Inventors: Sean P. Conlon, Loveland, OH (US); Kempton K. Carroll, II, Cincinnati, OH (US); James T. Spivey, Cincinnati, OH (US); Ragae M. Ghabrial, Loveland, OH (US)

(21) Appl. No.: 12/873,829

(22) Filed: Sep. 1, 2010

(51) Int. Cl. A61B 1/00

(52) U.S. Cl. 600/104

(57) ABSTRACT

A single port surgical method comprises inserting only a single trocar through the abdominal wall; inserting a camera into the abdominal cavity; magnetically anchoring the camera to the abdominal wall; obtaining a first instrument comprising an elongate shaft with a distal end and a proximal end connected to a first handle; passing the distal end of the first instrument through the abdominal wall independent of a trocar; attaching in vivo an end effector to the distal end of the first surgical instrument; obtaining a second instrument comprising an elongate shaft with a distal end with an end effector and a proximal end connected to a second handle; passing the distal end of the second instrument through the trocar; and manipulating tissue in the abdominal cavity with the first and second surgical instrument end effectors under visualization from the magnetically anchored camera.

### BACKGROUND

The present invention relates in general to surgical devices and procedures, and more particularly to minimally invasive surgery.

Surgical procedures are often used to treat and cure a wide range of diseases, conditions, and injuries. Surgery often requires access to internal tissue through open surgical procedures or endoscopic surgical procedures. Laparoscopic surgery has numerous advantages compared to traditional open surgical procedures, including reduced trauma, faster recovery, reduced risk of infection, and reduced scarring. Laparoscopic surgery is often performed with an insufflatory fluid present within the body cavity, such as carbon dioxide or saline, to provide adequate space to perform the intended surgical procedures. The insufflated cavity is generally under pressure and is sometimes referred to as being in a state of pneumoperitoneum. Surgical access ports are often used to facilitate surgical manipulation of internal tissue while maintaining pneumoperitoneum. For example, trocars are often used to provide a port through which laparoscopic surgical instruments are passed. Access ports generally have an instrument seal, which prevents the insufflatory fluid from escaping while an instrument is positioned in the port.

### BRIEF DESCRIPTION OF DRAWINGS

While the specification concludes with claims which particularly point out and distinctly claim the invention, it is believed the invention will be better understood from the following description taken in conjunction with the accompanying drawings illustrating some non-limiting examples of the invention. Unless otherwise indicated, the figures are not necessarily drawn to scale, but rather to illustrate the principles of the invention.

FIG. 1 depicts an example of traditional laparoscopic surgery;

FIG. 2 depicts an example of single port surgery;

FIG. 3 depicts an example of a single port surgical kit;

FIG. 4 depicts an example of a single port surgical kit;

FIG. 5 depicts an example of a single port surgical kit;

FIG. 6 depicts an example of a single port surgical kit;

FIG. 7 depicts an example of a single port surgical kit;

FIG. 8 depicts an example of a single port surgical kit; and

FIG. 9 depicts an example of a single port surgical kit.

### SUMMARY

A single port surgical method comprises inserting only a single trocar through the abdominal wall; inserting a camera into the abdominal cavity; magnetically anchoring the camera to the abdominal wall; obtaining a first instrument comprising an elongate shaft with a distal end and a proximal end connected to a first handle; passing the distal end of the first instrument through the abdominal wall independent of a trocar; attaching in vivo an end effector to the distal end of the first surgical instrument; obtaining a second instrument comprising an elongate shaft with a distal end with an end effector and a proximal end connected to a second handle; passing the distal end of the second instrument through the trocar; and manipulating tissue in the abdominal cavity with the first and second surgical instrument end effectors under visualization from the magnetically anchored camera.

In one variant the method may further comprises obtaining a third instrument comprising an elongate shaft with a distal end and a proximal end connected to a third handle; passing the distal end of the third instrument through the abdominal wall independent of a trocar; attaching in vivo an end effector to the distal end of the third surgical instrument; and manipulating tissue in the abdominal cavity with the first, second, and third surgical instrument end effectors under visualization from the magnetically anchored camera.

In another variant, the step inserting only a single trocar through the abdominal wall is performed through the patient's umbilicus.

The camera may comprises a tether and the trocar comprises an instrument cannula, and wherein inserting a camera into the abdominal cavity comprises threading the tether through the umbilicus but outside the trocar cannula.

In some examples, the step of manipulating tissue comprises performing one of an appendectomy, a cholecystectomy, a sleeve gastrectomy, a gastric bypass, or a greater curvature plication.

In an embodiment, a surgical kit comprises two percutaneous instruments each having an elongate shaft with a distal end and a proximal end connected to a handle; at least two end effectors attachable and detachable to the distal ends of the percutaneous instruments; a laparoscopic loader adapted to receive the end effectors; a trocar; and a laparoscopic surgical stapler. The surgical kit may further comprise laparoscopic energy based clamping and coagulating device and/or a laparoscopic suture device. Optionally, the surgical kit may further comprised a third percutaneous instrument having an elongate shaft with a distal end and a proximal end connected to a handle, and at least three end effectors attachable and detachable to the distal ends of the percutaneous instruments. The surgical kit may further comprised a magnetic camera and/or a magnetic external control unit adapted to anchor a magnetic camera. The components of the surgical kit may be contained in a sterile sealed package.

In another embodiment a surgical kit comprises two percutaneous instruments each having an elongate shaft with a distal end and a proximal end connected to a handle; at least two end effectors attachable and detachable to the distal ends of the percutaneous instruments; a laparoscopic loader adapted to receive the end effectors; a trocar; a laparoscopic clip applier; and a laparoscopic specimen retrieval device. Optionally the surgical kit may further comprises a magnetic camera and/or a magnetic external control unit adapted to anchor a magnetic camera.

In another embodiment, a sterile sealed surgical kit comprises two percutaneous instruments each having an elongate shaft with a distal end and a proximal end connected to a handle; at least two end effectors attachable and detachable to the distal ends of the percutaneous instruments; a laparoscopic loader adapted to receive the end effectors; a trocar; and a laparoscopic energy based clamping and coagulating device. Optionally, the kit may further comprising a laparoscopic suture device. The surgical kit may also comprises a magnetic camera and/or an magnetic external control unit adapted to anchor a magnetic camera.

### DETAILED DESCRIPTION

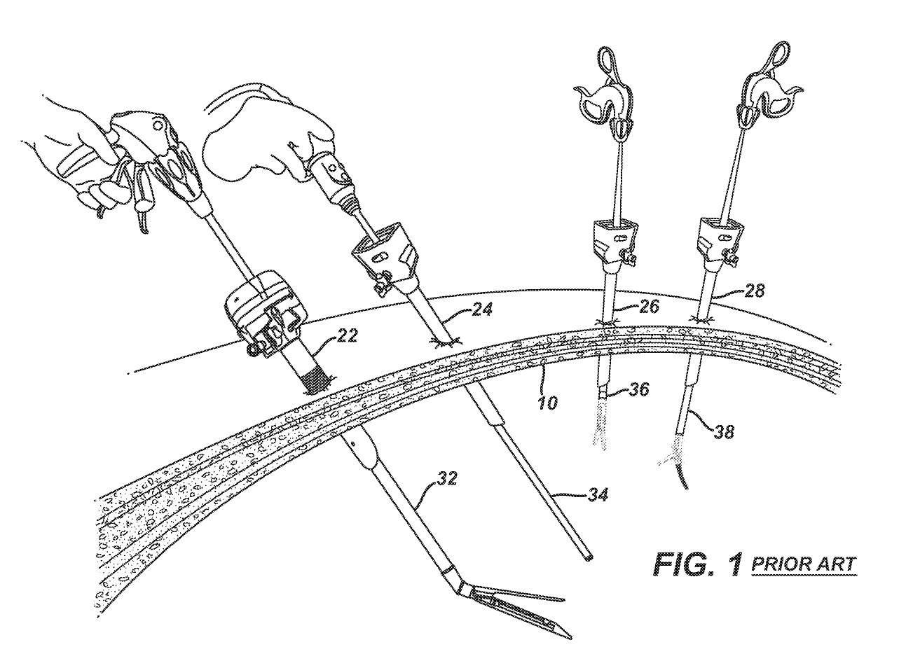

FIG. 1 depicts an example of traditional laparoscopic surgery. The abdominal cavity may be insufflated using a varess needle or through a trocar. A plurality of access ports, shown here as trocars (22, 24, 26, 28), are inserted through the abdominal wall (10). Each port (22, 24, 26, 28) accommodates a separate surgical device. In this example, the surgical devices include a surgical stapler (32), a laparoscope (34), shears (36), and a Maryland dissector (38). The laparoscope (34) is used to visualize the abdominal cavity, while the remaining instruments (32, 36, 38) are used to manipulate tissue.

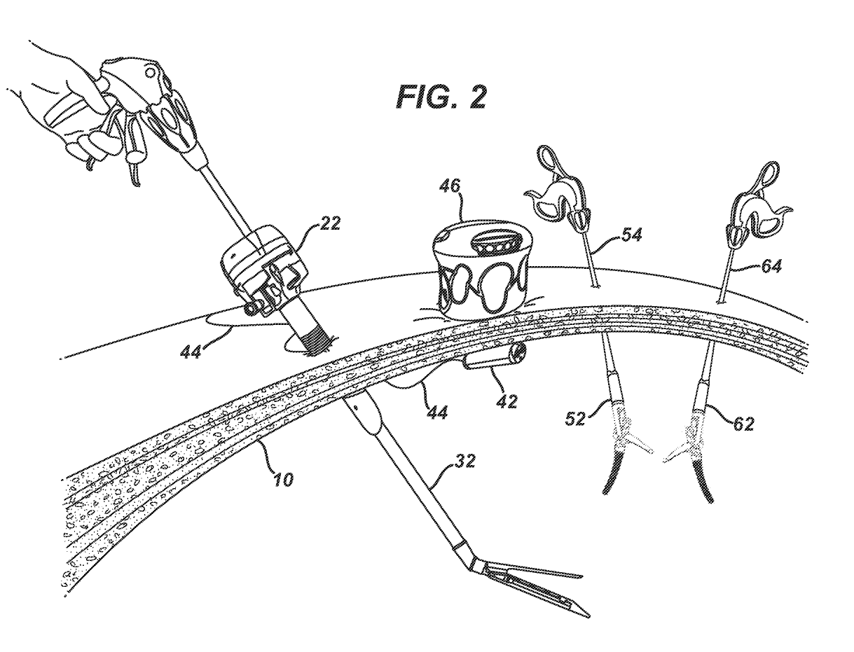

FIG. 2 depicts an example of an improved minimally invasive surgical technique. Unlike traditional laparoscopic surgery, only a single access port (22) is inserted through the abdominal wall (10). The port location may be in the patient's umbilicus; however, other locations could also be used. A camera (42) is inserted into the abdominal cavity, usually through an incision in the umbilicus. In the present example the camera (42) includes a tether (44) to provide power and data transmission; however, battery powered wireless cameras could also be used. The tether may be threaded through the access port (22) instrument cannula, or as shown here threaded the tether through the umbilicus but outside the trocar cannula an external control unit (46) magnetically anchors the camera (42) to the abdominal wall (10). The surgeon may change the location and orientation of the camera (42) by simply moving and rotating the external control unit (46). Examples of some external control units (46) and magnetically anchored cameras (42) are described in US patent publication numbers 20050165449, 20070255273, 20070255100, and 20050288555.

One or more percutaneous instruments (54, 64) each comprise an elongate shaft with a distal end and a proximal end connected to a handle. A variety of different end effectors (52, 62) can be attached and detached in vivo to the distal ends of the instruments (54, 64), respectively. A loader (not shown) can be passed through the access port (22) to facilitate attaching and detaching the end effectors (52, 62). The distal ends each directly puncture and pass through the abdominal wall (10) independent of a trocar or other access port, and the resilient abdominal wall (10) seals directly against the instrument shafts (54, 64) to maintain pneumoperitoneum. The instrument shafts (54, 64) have very small diameters compared to traditional laparoscopic instruments leaving little, if any, postoperative scarring or patient discomfort. Examples of some suitable percutaneous instruments (54, 64) and loaders are described in U.S. application Ser. Nos. 12/576,546, 12/576,565, 12/576,578, and U.S. Pat. Nos. 5,441,059 and 5,352,219.

During surgery, traditional laparoscopic surgical instruments may be used through the access port (22). As is well known to one with ordinary skill in the art, traditional laparoscopic instruments typically have an elongate shaft with a distal end with an end effector and a proximal end connected to a second handle. Shown in this example is an articulated surgical staplers, but a variety of other laparoscopic instruments could also be used such as specimen retrieval devices, suture devices, energy based clamping and coagulating devices, clip appliers, and the like.

The surgeon may conduct minimally invasive surgical procedures used one or more of the percutaneous instruments (54, 64) with attached end effectors (52, 62) in combination with a traditional laparoscopic instruments (32) under visualization from the magnetically anchored camera (42). Some non-limiting examples of such procedures include an appendectomy, a cholecystectomy, a sleeve gastrectomy, a gastric bypass, a greater curvature plication, and the like. One advantage of this minimally invasive surgical technique is that only a single access device (22) can be employed, thus reducing the number and size of incisions and reducing the attendant post operative scarring and pain. The magnetically anchored camera (42) eliminates the need for a dedicated access port normally required during traditional laparoscopic surgery, and further provides more options for the surgeon to change the camera's perspective during surgery. Still another advantage is that a traditional trocar may be used (typically 12 mm in size or less), which is smaller in diameter than other single port surgical options, thus reducing the size of the incision and attendant postoperative scarring and pain. Smaller ports also reduce the risk of herniation and the need for fascia closure. In yet another advantage, the spacing of the instruments (32, 54, 64) provide superior triangulation compared to other single port surgical options that try to pass multiple instrument simultaneously through the same port, thus facilitating simplifying access to tissue and improving surgeon leverage and comfort during surgery.

### Example 1

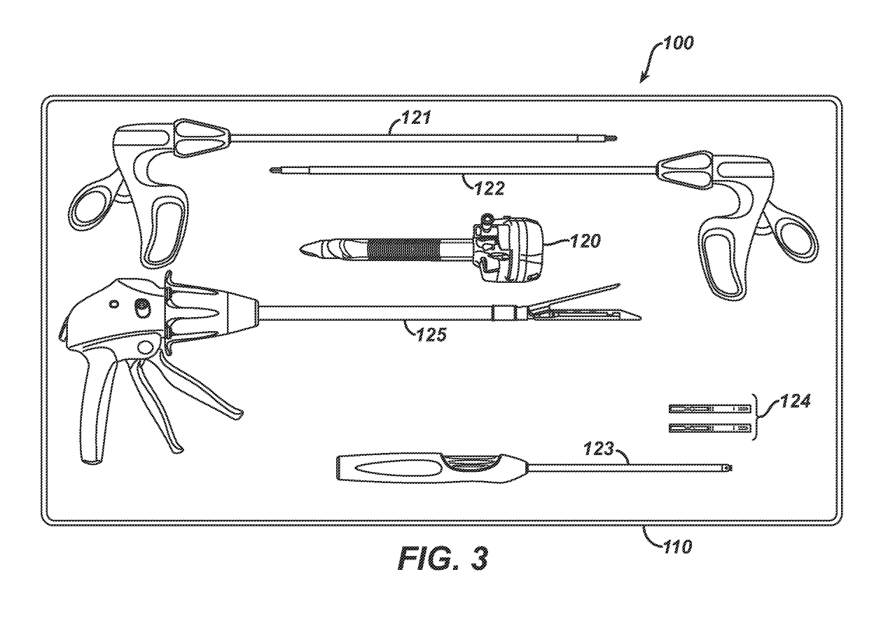

FIG. 3 illustrates an example of a minimally invasive surgical kit (100). The components of the kit (100) are preferably organized in a tray (110) and stored in a sterile sealed package. In this example the kit (100) includes a 12 mm trocar (120), two percutaneous instruments (121, 122), a loader (123), and two or more end effectors (124) attachable to percutaneous instruments. The end effectors (124) may include a grasper and a Maryland dissector (but other end effectors could also be used) and may be preloaded in sleeves for easy attachment to the loader (123). The kit (100) further includes a traditional laparoscopic surgical stapler (125), such as the ECHELON or ENDO GIA endocutters. The kit (100) may optionally include a camera (42) and/or an external control unit (46), which would be particularly beneficial if either of those components were disposable.

The kit (100) may be used for a variety of surgical procedures. The following describes one representative surgical technique involves an appendectomy. The trocar (120) is inserted at the right upper quadrant and then removed. The camera (42) is inserted through the incision and then the trocar (120) is reinserted such that the tether (44) is outside the trocar (120) cannula. The camera (42) may be positioned using the external control unit (46) to visualize the surgical site. Using the loader (223) and end effectors (224), a percutaneous grasper (121) is assembled through the umbilicus and a percutaneous Maryland dissector (122) is assembled at the midline approximately 2-3 cm below the umbilicus.

The surgeon may next expose the Appendix. Using the percutaneous instruments (121, 122), the cecum is retracted upward toward the liver, elevating the appendix in the optical field of the camera (42). The appendix may be grasped with a 5 mm claw-type grasper inserted via the supra-pubic trocar (120), and the appendix is held toward abdominal wall (10). The 5 mm claw-type grasper may optionally be included in the kit (100). Next, the surgeon may create the mesenteric window. Using the percutaneous Maryland dissector (122), mesenteric window may be created under the base of the appendix. The window may be made as close as possible to the base of the appendix, approximately 1 cm in size.

The surgeon may now transect the mesoappendix and appendix. The appendix is transected by inserting the surgical stapler (125) via the trocar (120), closing it around the base of the appendix and firing it. The base of the appendix is inspected for hemostasis. The surgical stapler (125) cartridge may be changed to a vascular cartridge and the mesoappendix is transected with the same instrument. Several cartridges may be used. The kit (100) may optionally include the reload cartridges for the surgical stapler (125). The appendix is now amputated from the gastrointestinal tract. A specimen retrieval bag, which may optionally be included with the kit (100) is inserted via the trocar (120) and deployed in the intra-abdominal cavity. The appendix, held by the percutaneous grasper (121), is placed into the specimen bag. The bag is closed and the specimen bag is removed with the trocar (120) from the intra-abdominal cavity. The specimen bag is separated from the trocar (120), and the trocar is reinserted.

The intra-abdominal cavity may be irrigated thoroughly with normal saline. For perforated appendicitis with or without an intra-abdominal abscess, a Blake Drain may be left in the right lower quadrant and pelvis.

### Example 2

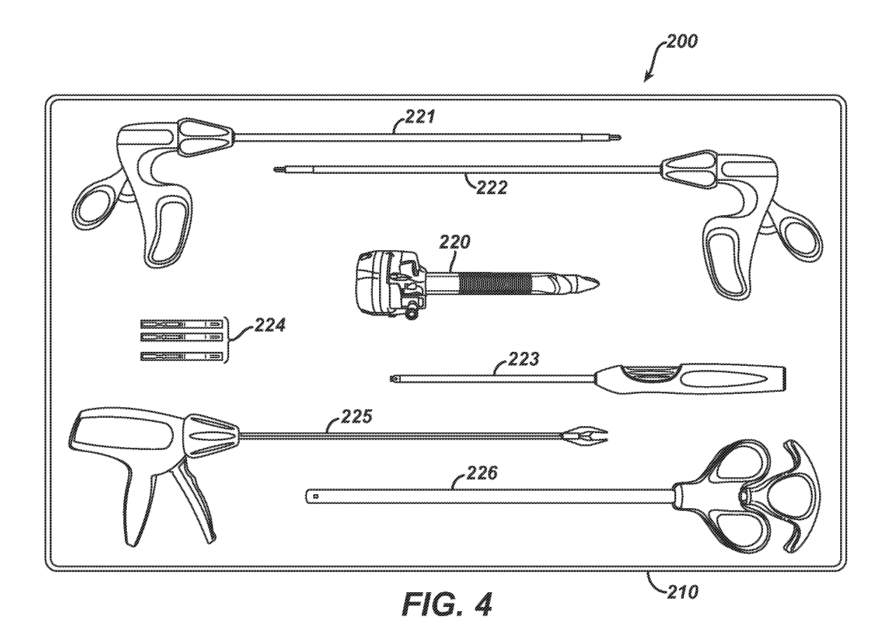

FIG. 4 illustrates another example of a minimally invasive surgical kit (200). The components of the kit (200) are preferably organized in a tray (210) and stored in a sterile sealed package. In this example the kit (200) includes a 12 mm trocar (220), two percutaneous instruments (221, 222), a loader (223), and three or more end effectors (224) attachable to percutaneous instruments. The end effectors (224) may include graspers and a Maryland dissector (but other end effectors could also be used) and may be preloaded in sleeves for easy attachment to the loader (223). The kit (200) further includes a traditional laparoscopic clip applier (225), such a LIGAMAX or ENDO CLIP clip appliers. The kit (200) further includes a traditional laparoscopic specimen retrieval device (226), such an ENDOPOUCH or ENDOBAG specimen retrievers. The kit (200) may optionally include a camera (42) and/or an external control unit (46).

The kit (200) may be used for a variety of surgical procedures. The following describes one representative surgical technique involves a cholecystectomy. The surgeon may access the abdomen by create incision the umbilicus, inserting the trocar (220), and then establishing pneumoperitoneum. The trocar (220) may then be removed and the camera (42) inserted through the incision and then attracted to the body wall (10) using external control unit (46). The camera (42) can now be manipulated into the desired viewing position. The trocar (220) may be reinserted alongside the tether (44). One percutaneous instrument (221) is inserted through the abdominal wall (10) at the right anterior axillary line along the costal margin, and one percutaneous instrument (222) is inserted in the epigastric region in direct line with the infidibulum. The loader (223) is used through the trocar (220) to assemble grasping end effectors (224) onto the percutaneous instruments (221, 222).

The surgeon may now expose the surgical site by grasping the fundus of gallbladder with percutaneous instrument (221) and push gallbladder over the liver toward the right shoulder. The second percutaneous instrument (222) is placed on the base of the gallbladder and retracts anteriorly to expose Calot's triangle.

The surgeon may now skeletonize the Calot's triange using a Maryland dissector through the 12 mm trocar (which may optionally be included in the kit (200)). The surgeon may now transect the cystic duct by skeletonizing the cysctic duct using the dissector, clip the duct using the clip applier (225), and then transects the cystic duct with laparoscopic scissors (which may optionally be included in the kit (200) or as an end effector (224)).

The cystic artery may be ligated by skeletonizing cystic artery using blunt dissection with a dissector. A clip is applied over the cystic artery using the clip applier (225). The cystic artery may be transected with the scissors. The gallbladder now may be dissected by grasping Hartman's pouch and retracting anteriorally using one of the percutaneous graspers (221, 222). The surgeon may now dissect between liver and gall bladder with HARMONIC dissector. The specimen is removed using the specimen retrieval device (226).

### Example 3

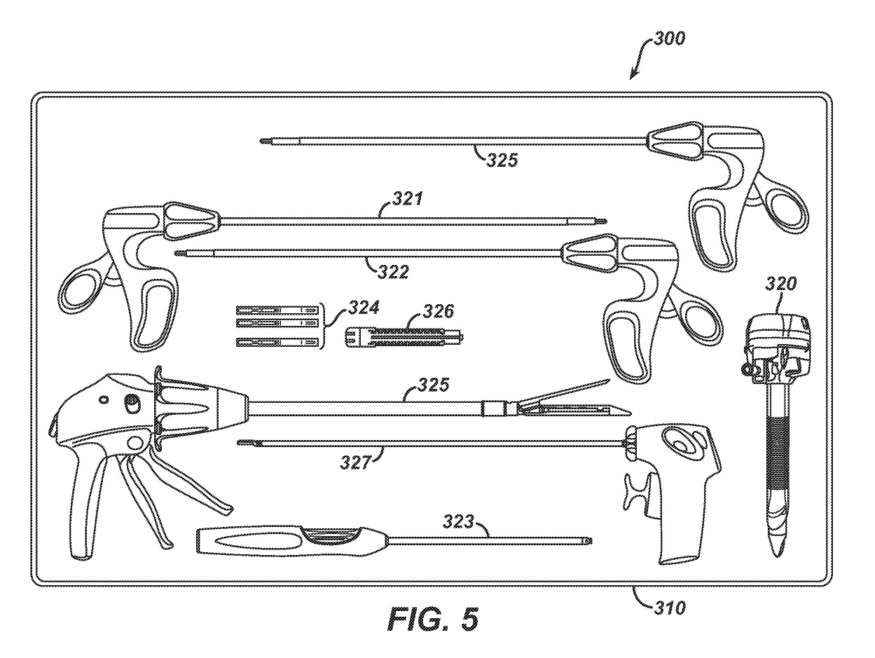

FIG. 5 illustrates an example of a minimally invasive surgical kit (300). The components of the kit (300) are preferably organized in a tray (310) and stored in a sterile sealed package. In this example the kit (300) includes a 12 mm trocar (320), three percutaneous instruments (321, 322, 329), a loader (323), and two or more end effectors (324) attachable to percutaneous instruments. The end effectors (324) may include graspers and a babcock (but other end effectors could also be used) and may be preloaded in sleeves for easy attachment to the loader (323). The kit (300) further includes a traditional laparoscopic surgical stapler (325) with cartridge reloads (326). The kit (300) also includes an energy based clamping and coagulating device (327), such as an ACE ultrasonic shears or LIGASURE bi-polar hemostatic tool. The kit (300) may optionally include a camera (42) and/or an external control unit (46).

The kit (300) may be used for a variety of surgical procedures. The following describes one representative surgical technique involves a sleeve gastrectomy. Insufflate the abdominal cavity using a varess needle (optionally be included in the kit (300)) at the umbilicus. Remove the varess needle and insert the trocar (320) through the umbilicus (Site 1). Remove the trocar (320) and place the camera (42) through the incision site. Attach the camera (42) to the external control unit (46) and move camera to desired position. Re-insert the trocar (320) next to the tether (44).

Insert percutaneous instrument (321) through the wall (10) in the lower right quadrant. Insert the loader (323) with a grasping end effector (324) through the trocar (320) and attach to it to the percutaneous instrument (321) (Site 2). Next insert percutaneous instrument (322) in the lower left quadrant, insert the loader (323) with a grasping end effector (324) and attach it percutaneous instrument (322) (Site 3). Next insert one percutaneous instrument (329) in the upper right quadrant, insert the loader (323) with a grasping end effector (324) and attach it percutaneous instrument (329) (Site 4).

The surgeon may now mobilizes the greater curvature of the stomach by retracting the liver using site 2 grasper, manipulating tissue using site 3 grasper, creating tension on tissue using site 4 grasper, and dissecting the omentum using energy device (327) through site 1. Blunt dissect to skeletonize vessels using site 4 grasper, and dissect vessels using harmonic device through site 1. Ligate vessels as needed through site 1.

The surgeon may now locate the start point and begin transaction. The surgeon may insert bougie to facilitate the procedure. After locating the pylorus, measure about 2-6 cm from the pylorus to locate initial site for stapling. Use site 1 trocar (320) and insert the surgical stapler (325). Using the grasper in site 3, manipulate the stomach into the surgical stapler (325) jaws and fire stapler parallel to lesser curvature of the stomach. Aim towards the lessor curve and leave a distance of about 2-3 cm. Complete 2nd and third firings of the stapler (325) and then remove the bougie. The surgeon may inspect staple line, and line if needed by oversewing.

The surgeon may now remove the resected portion of the stomach. Final inspection of the anastomosis by using air, and introduce a drain tube through trocar (320) site 1. Place drain tube along the greater curvature of the stomach. The distal end of the drain is placed at the angle of His. Fix the drain tube using suture to prevent migration. Using trocar (320) site 1, insert a specimen retrieval bag (optionally included in the kit (300), and place stomach specimen in bag using appropriate percutaneous grasper. Remove specimen and trocar (320) from the umbilicus site.

### Example 4

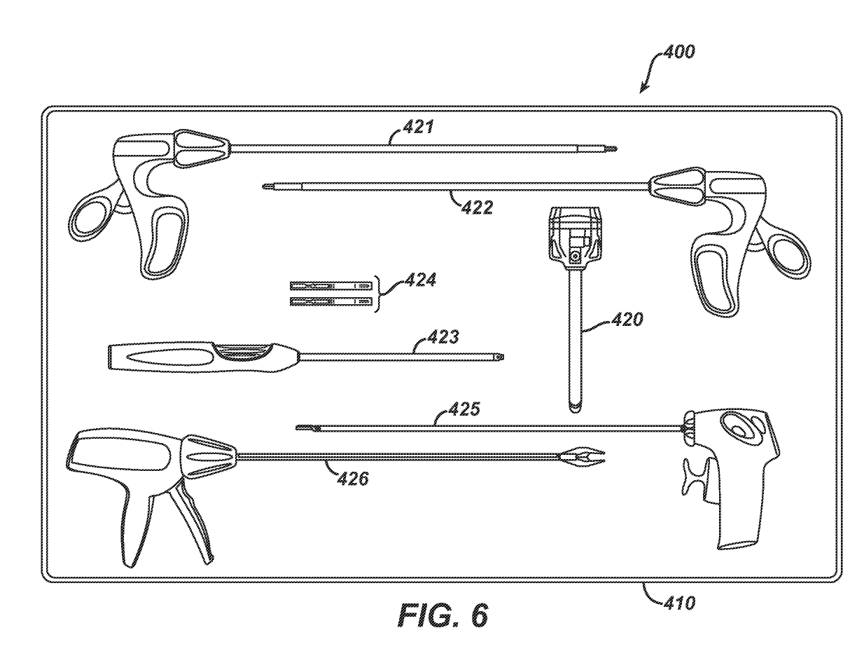

FIG. 6 illustrates an example of a minimally invasive surgical kit (400). The components of the kit (400) are preferably organized in a tray (410) and stored in a sterile sealed package. In this example the kit (400) includes a 5 mm trocar (420), two percutaneous instruments (421, 422), a loader (423), and two or more end effectors (424) attachable to percutaneous instruments. The end effectors (424) may include graspers (but other end effectors could also be used) and may be preloaded in sleeves for easy attachment to the loader (423). The kit (400) includes an energy based clamping and coagulating device (425). The kit (400) also includes a multifeed stapler (426), such the device described in US patent publication 20100187284. The kit (400) may optionally include a camera (42) and/or an external control unit (46).

The kit (400) may be used for a variety of surgical procedures. The following describes one representative surgical technique involves a greater curvature plication. The surgeon may accesses the abdomen by create incision at the umbilicus, inserting the trocar (420), and establishing pneumoperitoneum. The trocar (420) may then be removed and the camera (42) inserted through the incision and then attracted to the body wall (10) using external control unit (46). The camera (42) can now be manipulated into the desired viewing position. The trocar (420) may be reinserted alongside the tether (44). One percutaneous instrument (421) is inserted through the abdominal wall (10) at the patient's right upper quadrant, and one percutaneous instrument (422) is inserted at the patient's right left quadrant. The loader (423) is used through the trocar (420) to assemble grasping end effectors (424) onto the percutaneous instruments (421, 422).

The surgeon may now separate the greater omentum and short gastric vessels from greater curvature by grasping antrum of stomach with one of the percutaneous graspers (421, 422) and separate greater omentum from gastric wall near the greater curvature using the energy device (425). The second percutaneous grasper may be used for counter traction along the body and fundus of the stomach to aid in this dissection. The dissection may be performed along the greater curvature to the angle of His and includes the short gastric vessels. The dissection may also be performed along the greater curvature to within 2-6 cm from the pylorus.

The surgeon next creates a plication using multiple rows of staples. A bougie may be inserted in the patient to facilitate creating a plication. The multifeed stapler (426) is inserted through the trocar (420). Using the percutaneous graspers (421, 422), the surgeon may create access for the multifeed stapler (426) to create a staple line that extends from within 1-2 cm of the angle of His to about 2-6 cm of the pylorus. This staple line secures anterior and posterior surfaces of the stomach creating a plication or otherwise imbricating the greater curvature of the stomach along this length. Staple bites may be generally centered about the greater curvature. Spacing between staples may be up to 3 cm. The process may be repeated creating a second row about the first resulting in a larger plication of tissue. The process may be repeated as needed until desired stomach shape is achieved. In the outermost row, spacing between staples may be roughly 1 cm.

### Example 5

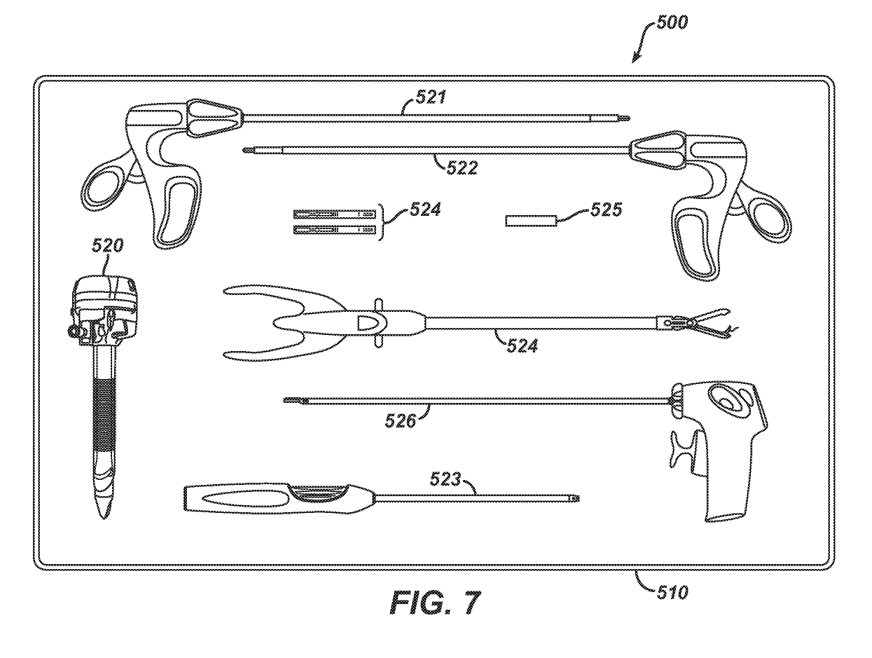

FIG. 7 illustrates an example of a minimally invasive surgical kit (500). The components of the kit (500) are preferably organized in a tray (510) and stored in a sterile sealed package. In this example the kit (500) includes a 12 mm trocar (520), two percutaneous instruments (521, 422), a loader (523), and two or more end effectors (524) attachable to percutaneous instruments. The end effectors (524) may include graspers (but other end effectors could also be used) and may be preloaded in sleeves for easy attachment to the loader (523). The kit (500) includes a laparoscopic suture device (524) with reloads (525), such as an ENDO STITCH. The kit (500) also includes an energy based clamping and coagulating device (526). The kit (500) may optionally include a camera (52) and/or an external control unit (56).

The kit (500) may be used for a variety of surgical procedures. The following describes one representative surgical technique involves a greater curvature plication. The surgeon may accesses the abdomen by create incision at the umbilicus, inserting the trocar (520), and establishing pneumoperitoneum. The trocar (520) may then be removed and the camera (52) inserted through the incision and then attracted to the body wall (10) using external control unit (56). The camera (52) can now be manipulated into the desired viewing position. The trocar (520) may be reinserted alongside the tether (54). One percutaneous instrument (521) is inserted through the abdominal wall (10) at the patient's right upper quadrant, and one percutaneous instrument (522) is inserted at the patient's right left quadrant. The loader (523) is used through the trocar (520) to assemble grasping end effectors (524) onto the percutaneous instruments (521, 422).

The surgeon may now separate the greater omentum and short gastric vessels from greater curvature by grasping antrum of stomach with one of the percutaneous graspers (521, 522) and separate greater omentum from gastric wall near the greater curvature using the energy device (525). The second percutaneous grasper may be used for counter traction along the body and fundus of the stomach to aid in this dissection. The dissection may be performed along the greater curvature to the angle of His and includes the short gastric vessels. The dissection may also be performed along the greater curvature to within 2-6 cm from the pylorus.

The surgeon next creates a plication using multiple rows of sutures. A bougie may be inserted to facilitate creating a gastric plication. The suture device (524) is inserted through the trocar (520). Using both percutaneous graspers (521, 522), the surgeon may create access for suturing device (524) to create a running or interrupted suture line that extends from about 1-2 cm of the angle of His to within about 2-6 cm of the pylorus. This suture line may involve alternating bites on the anterior and posterior surfaces of the stomach creating a plication or otherwise imbricating the greater curvature of the stomach along this length. Suture bites may be generally centered about the greater curvature. The process may be repeated to create a second row about the first resulting in a larger plication of tissue. The process may be repeated as needed until desired stomach shape is achieved.

### Example 6

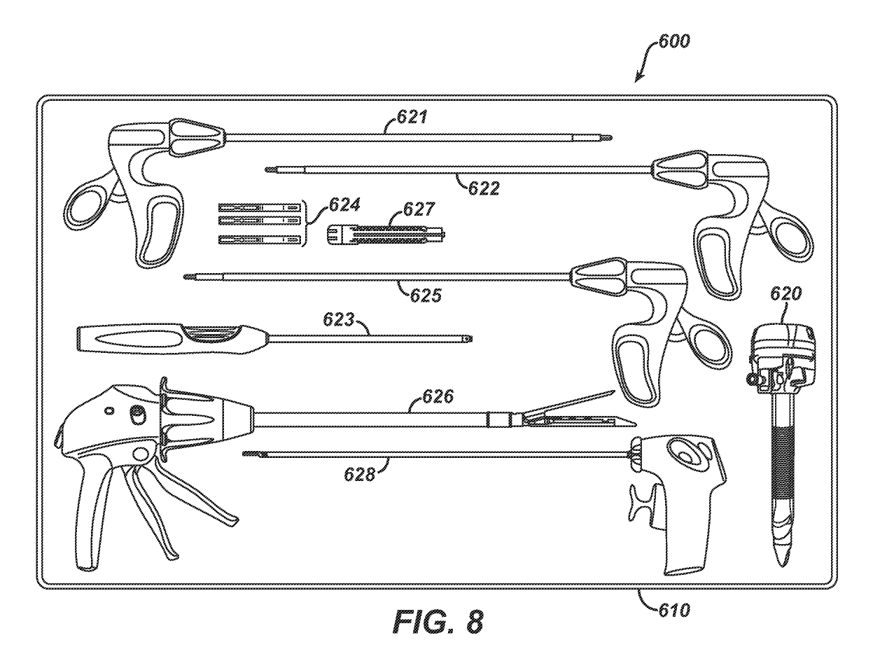

FIG. 8 illustrates an example of a minimally invasive surgical kit (600). The components of the kit (600) are preferably organized in a tray (610) and stored in a sterile sealed package. In this example the kit (600) includes a 12 mm trocar (620), three percutaneous instruments (621, 622, 625), a loader (623), and three or more end effectors (624) attachable to percutaneous instruments. The end effectors (624) may include graspers and scissors (but other end effectors could also be used) and may be preloaded in sleeves for easy attachment to the loader (623). The kit (300) further includes a laparoscopic surgical stapler (626) with cartridge reloads (627). The kit (300) also includes an energy based clamping and coagulating device (628). The kit (600) may optionally include a camera (62) and/or an external control unit (66).

The kit (600) may be used for a variety of surgical procedures. The following describes one representative surgical technique involves a gastric bypass. The surgeon may accesses the abdomen by create incision at the umbilicus, inserting the trocar (620), and establishing pneumoperitoneum. The trocar (620) may then be removed and the camera (62) inserted through the incision and then attracted to the body wall (10) using external control unit (66). The camera (62) can now be manipulated into the desired viewing position. The trocar (620) may be reinserted alongside the tether (64). One percutaneous instrument (621) is inserted through the abdominal wall (10) at the patient's right side, and one percutaneous instrument (622) is inserted at the patient's right left side. The loader (623) is used through the trocar (620) to assemble grasping end effectors (624) onto the percutaneous instruments (621, 622).

By steering the camera (42) and manipulating tissue with the percutaneous instruments (621, 622), the surgeon may examine the abdomen, including the liver and lesser sac, to determine feasibility of the operating and identify any obstructions. The surgeon may access the intestine to identifies obstructing adhesions, and dissect as needed. The omentum may be lifted and mobilized.

The surgeon may next create a jejuno-jejunal (J-J) anastomosis. The surgeon may identify the Ligament of Treitz, and using the percutaneous instruments (621, 622) to dissect and make a hole in transverse colon mesentery (preferably retrocolic only). The surgeon may landmark or note position of the mesenteric hole. Using the surgical stapler (626) through the trocar (620), the surgeon may transects the bowel about 20-40 cm from Ligament of Treitz. Using the energy device (628) through the trocar (620), the surgeon may transected additional mesentery to free the bowel.

With the percutaneous instruments (621, 622), the surgeon may pull and measure out bowel for Roux Limb (about 75-200 cm). Optionally, the surgeon may place a suture “pin” biliary to distal limb. Using scissors the surgeon can makes otomies in biliary and distal limbs. The surgical stapler (626) may be inserted in to the otomies and fired. Sutures may be used to close the otomies. Repair length of mesentery on previously mobilized Roux Limb. Using percutaneous graspers, the surgeon may transports bowel towards the lesser sac (and through mesenteric hole, if retrocolic). Suture mesentery closed around and into Roux Limb (if retrocolic).

The surgeon may now make a gastric pouch. The liver is retracted liver using percutaneous graspers and local anesthesia is injected. Using scissors the surgeon may make an incision and insert a Nathanson Liver Retractor. Alternatively, the surgeon may use the third percutaneous instrument (625) to retract the liver. The surgeon now identifies and accesses GE junction, spleen, lesser curve of stomach, and lesser sac (area posterior to stomach). The surgeon may estimate about 4-6 cm inferior to GE junction, and using the percutaneous instruments (621, 622) dissect fat to make window in lesser omentum at the lesser curve and remove fat pad from GE junction. Using the surgical stapler (626) trough the trocar (620), the surgeon may staple across lesser curve and use a guiding tool for stapling into position.

The surgeon may now connect the Roux Limb to the pouch. Using the percutaneous instruments (621, 622) the surgeon may bring the Roux Limb ante-gastric near pouch and place sutures to “pin” Limb to pouch. Using scissors the surgeon may make an otomy in pouch against form of E-Z tube (about 12 mm o.d.), and make a second otomy in Roux Limb of a size proportional to first otomy. The surgeon may then suture the interior layer and exterior layer of anastomosis around E-Z tube, and apply glue (or omentum patch) to the anastomosis.

### Example 7

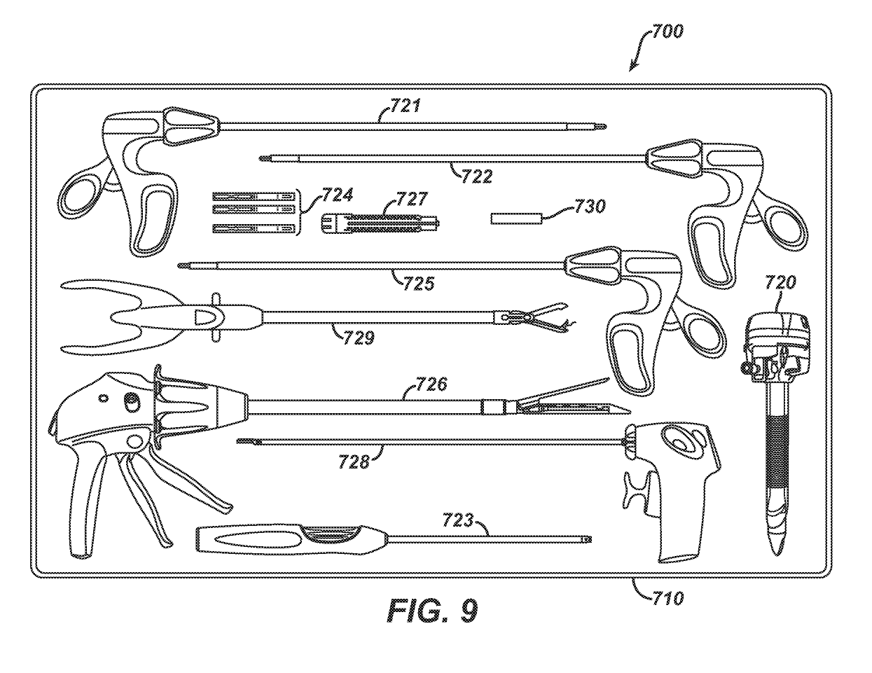

FIG. 9 illustrates an example of a minimally invasive surgical kit (700). The components of the kit (700) are preferably organized in a tray (710) and stored in a sterile sealed package. In this example the kit (700) includes a 12 mm trocar (720), three percutaneous instruments (721, 622, 625), a loader (723), and three or more end effectors (724) attachable to percutaneous instruments. The end effectors (724) may include graspers and a babcock (but other end effectors could also be used) and may be preloaded in sleeves for easy attachment to the loader (723). The kit (300) further includes a laparoscopic surgical stapler (726) with cartridge reloads (727). The kit (300) also includes an energy based clamping and coagulating device (728). The kit further includes a laparoscopic suture device (729) and reloads (730). The kit (700) may optionally include a camera (62) and/or an external control unit (66).

The kit (700) may be used for a variety of surgical procedures. One representative surgical technique involves a gastric bypass and employs a method similar to the gastric bypass described in example 6; however, the suture device (729) may be used to facilitate throwing sutures.

Having shown and described various embodiments and examples of the present invention, further adaptations of the methods and devices described herein can be accomplished by appropriate modifications by one of ordinary skill in the art without departing from the scope of the invention. Several of such potential modifications have been mentioned, and others will be apparent to those skilled in the art. For instance, the specific materials, dimensions, devices, and the scale of drawings will be understood to be non-limiting examples. Additionally, the surgical techniques described above are described are provided as an overview, and various methods and devices known in the art may be used to augment the techniques. Accordingly, the scope of the present invention should be considered in terms of the following claims and is understood not to be limited to the details of structure, materials, or acts shown and described in the specification and drawings.

1. A single port surgical method, comprising:
inserting only a single trocar through the abdominal wall;
inserting a camera into the abdominal cavity;
magnetically anchoring the camera to the abdominal wall;
obtaining a first instrument comprising an elongate shaft with a distal end and a proximal end connected to a first handle;
passing the distal end of the first instrument through the abdominal wall independent of a trocar;
attaching in vivo an end effector to the distal end of the first surgical instrument;
obtaining a second instrument comprising an elongate shaft with a distal end with an end effector and a proximal end connected to a second handle;
passing the distal end of the second instrument through the trocar; and
manipulating tissue in the abdominal cavity with the first and second surgical instrument end effectors under visualization from the magnetically anchored camera.

2. The method of claim 1, further comprising:
obtaining a third instrument comprising an elongate shaft with a distal end and a proximal end connected to a third handle;
passing the distal end of the third instrument through the abdominal wall independent of a trocar;
attaching in vivo an end effector to the distal end of the third surgical instrument; and
manipulating tissue in the abdominal cavity with the first, second, and third surgical instrument end effectors under visualization from the magnetically anchored camera.

3. The method of claim 1, wherein the step inserting only a single trocar through the abdominal wall is performed through the patient's umbilicus.

4. The method of claim 3 wherein the camera comprises a tether and the trocar comprises an instrument cannula, and wherein inserting a camera into the abdominal cavity comprises threading the tether through the umbilicus but outside the trocar cannula.

5. The method of claim 1, wherein the step of manipulating tissue comprises performing an appendectomy.

6. The method of claim 1, wherein the step of manipulating tissue comprises performing a cholecystectomy.

7. The method of claim 1, wherein the step of manipulating tissue comprises performing a sleeve gastrectomy.

8. The method of claim 1, wherein the step of manipulating tissue comprises performing a gastric bypass.

9. The method of claim 1, wherein the step of manipulating tissue comprises performing a greater curvature plication.

10. A single port surgical method, comprising:
inserting only a single surgical access port through the abdominal wall;
inserting a camera into the abdominal cavity;
magnetically anchoring the camera to the abdominal wall;
obtaining a first instrument comprising an elongate shaft with a distal end and a proximal end connected to a first handle;
passing the distal end of the first instrument through the abdominal wall independent of a surgical access port;
attaching an end effector to the distal end of the first surgical instrument; and
obtaining a second instrument comprising an elongate shaft with a distal end with an end effector and a proximal end connected to a second handle;
passing the distal end of the second instrument through the surgical access port; and
manipulating tissue in the abdominal cavity with the first and second surgical instrument end effectors under visualization from the magnetically anchored camera.

11. The method of claim 10, wherein the step of manipulating tissue comprises performing one of an appendectomy, a cholecystectomy, a sleeve gastrectomy, a gastric bypass, or a greater curvature plication.

12. A single port surgical method, comprising:
inserting only a single surgical access port through the abdominal wall;
inserting a camera into the abdominal cavity;
magnetically anchoring the camera to the abdominal wall;
obtaining a first instrument comprising an elongate shaft with a distal end and a proximal end connected to a first handle;
passing the distal end of the first instrument through the abdominal wall independent of a surgical access port;
attaching in vivo an end effector to the distal end of the first surgical instrument; and
obtaining a second instrument comprising an elongate shaft with a distal end with an end effector and a proximal end connected to a second handle;
passing the distal end of the second instrument through the surgical access port; and
obtaining a third instrument comprising an elongate shaft with a distal end and a proximal end connected to a third handle;
passing the distal end of the third instrument through the abdominal wall independent of a trocar;
attaching in vivo an end effector to the distal end of the third surgical instrument; and
manipulating tissue in the abdominal cavity with the first, second, and third surgical instrument end effectors under visualization from the magnetically anchored camera.

13. The method of claim 12, wherein the step of manipulating tissue comprises performing one of an appendectomy, a cholecystectomy, a sleeve gastrectomy, a gastric bypass, or a greater curvature plication.
